(19) United States
(12) Patent Application Publication
(10) Pub. No.: US 2010/0279372 A1
(43) Pub. Date: Nov. 4, 2010

(54) METHOD OF PRETREATING LIGNOCELLULOSE-BASED BIOMASS

(75) Inventors: Hwa Young CHO, Hwaseong-si (KR); Sang Jun SIM, Suwon-si (KR); Jin Woo KIM, Bucheon-si (KR); Se Jong HAN, Gunpo-si (KR); Jae Chan PARK, Yongin-si (KR); Sung Min PARK, Yongin-si (KR)

Correspondence Address: CANTOR COLBURN, LLP, 20 Church Street, 22nd Floor, Hartford, CT, 06103, US

(73) Assignee: Samsung Electronics Co., Ltd., Suwon-si (KR)

(21) Appl. No.: 12/499,383

(22) Filed: Jul. 8, 2009

(30) Foreign Application Priority Data
Apr. 30, 2009 (KR) 10-2009-0037915

(51) Int. Cl. C13K 1/02; C12P 7/02; C12P 7/10; C08B 1/00

(52) U.S. Cl. 435/165; 127/37; 435/155; 536/56

(57) ABSTRACT

Disclosed is a method of pretreating lignocellulose-based biomass by extracting lignin from biomass by adding a solvent for dissolving lignin to the lignocellulose-based biomass including lignin, hemicellulose and cellulose, and extracting the cellulose and/or hemicellulose by adding an ionic liquid to the remaining biomass after extracting the lignin.

This application claims the benefit of Korean Patent Application Nos. 2009-37915, filed on Apr. 30, 2009, and all the benefits accruing therefrom under 35 U.S.C. §119, the contents of which in its entirety are herein is incorporated by reference.

### BACKGROUND

1. Field

The disclosure relates to a method of pretreating lignocellulose-based biomass and a method of producing biofuel using the same.

2. Description of Related Art

With globally increasing concern about exhaustion of resources and pollution of the environment by overuse of fossil fuels, the development of novel and renewable alternative energy sources that stably and continuously produce energy is being considered. As an example of this development of alternative energy, technology for producing energy from biomass has been attracting considerable attention.

Biomass includes saccharides generated by biosynthesis through carbon dioxide assimilation by fixing carbon dioxide using solar light, that is, photosynthesis. Biosynthetic saccharides may be produced by many types of living organisms. Lignocellulose is a representative example, of a plant source, which is rich, abundant and renewable.

Lignocellulose is a complex of a non-degradable aromatic polymer, lignin, and cellulose and hemicellulose as carbohydrates, and is called biomass in a narrow sense. Fuels produced from biomass are called biofuels. Biofuels can include hydrogen, diesel fuel and water soluble fuels such as alcohols.

Cellulose, a significant component of lignocellulose, is a stable polysaccharide having a linear chain of glucoses linked by β-1,4 glycosidic bonds. The cellulose has a more physically and chemically stable structure in a natural state than amylose, which has a spiral chain linked by α-1,4 glycosidic bonds.

Hemicellulose, another significant component of lignocelluloses, is a polysaccharide with a lower degree of polymerization than cellulose. Hemicellulose is a polymer of five-carbon monosaccharides, xyloses, or a polymer of a small quantity of five-carbon monosaccharides, arabinoses and six-carbon monosaccharides, such as mannoses, galactoses or glucoses. Because hemicellulose has a low degree of polymerization and less regular structure than cellulose, it is more easily degraded by physical and chemical treatments.

Lignin is a hydrophobic polymer with a complex structure and a high molecular weight. Lignin, in part contributes to the protection of plants from various biochemical attacks and external attacks, from microorganisms such as fungi, and insects. Because lignin is naturally and chemically robust, it is considered a material that is one of the least vulnerable to degradation among natural compounds existing in the natural world.

To produce various bioalcohols including ethanol or other compounds from lignocellulose, a polysaccharide component of lignocellulose may be converted into a fermentable saccharide to a concentration at which ethanol fermentation occurs.

In the production of biofuel, lignocellulose is usually pretreated to convert it into a fermentable saccharide. During the pretreatment, lignin and hemicellulose are partially removed, or the bond with cellulose becomes weakened and cellulose is partially degraded, resulting in easy approach of enzymes towards cellulose. The pretreatment of lignocellulose may be carried out through a physical, a chemical, a biological method or a combination of these.

Examples of the physical pretreatment methods can include a milling and a steam explosion. The milling method involves grinding a lignocellulose particle into very fine particles using a milling apparatus to induce a structural change. However, milling is not cost-effective due to high energy consumption and low efficiency. The steam explosion method involves steaming lignocellulose in a high-pressure container filled with high temperature steam for a predetermined period of time, and instantaneously releasing the pressure in the container to allow the structure of the lignocellose to be more accessible to enzymatic attack.

To improve the effects of the above-described physical methods, a physical-chemical method combining a physical method and a chemical method has been widely researched. For example, lignocellulose is hydrolyzed in 2% (w/w) or less sulfuric acid solution through dilute-acid hydrolysis, and steamed in a high-temperature vapor at about 160 to about 200° C. furfural, which acts as a fermentation inhibiting material.

Generally, the dilute-acid hydrolysis is a method of hydrolyzing hemicellulose to break bonds between cellulose, hemicellulose and lignin in lignocellulose, which results in facilitating enzymatic saccharification. As a result, a hydrolyte of hemicellulose, such as xylose dissolved in hydrolysis and saccharification solutions, may be obtained by fractionation, and insoluble cellulose and lignin which are not yet degraded by fractionation are converted into glucose and lignin residues through enzymatic saccharification. The lignin residue is transferred to subsequent fermentation processes.

An alternate method of fractionating biomass that utilizes a base, instead of an acid, is the ammonia fiber explosion (AFEX) developed by Bruce Dale et al. (Enzyme hydrolysis and ethanol fermentation of liquid hot water and AFEX pretreated distillers' grains at high-solids loadings (Bioresource Technology, Volume 99, Issue 12, August 2008, Pages 5206-5215. Youngmi Kim, Rick Hendrickson, Nathan S. Mosier, Michael R. Ladisch, Bryan Bals, Venkatesh Balan, Bruce E. Dale).

In the AFEX process, ammonia and a biomass are mixed in a ratio of 1:1 to 1:3, the resulting mixture is treated at a high temperature for about 5 to about 30 minutes, and the pressure of a reaction vessel containing the mixture is explosively released to atmospheric pressure to recycle gaseous ammonia and cause physical and chemical changes to the biomass structure, thereby improving the rate of enzymatic saccharification.

Unlike the dilute-acid hydrolysis, little hemicellulose is hydrolyzed, but most lignin is dissolved, thereby separating the lignin from cellulose and hemicellulose. Then, the cellulose and hemicellulose may be saccharified through subsequent enzymatic saccharification, such that five-carbon saccharides such as glucose and a pentose such as xylose may be obtained.

More recently, research into the possibility of commercializing ionic liquids as a media for extracting or dissolving cellulose from a woody biomass is ongoing. However, there is a limitation to the industrial application of the use of ionic liquids due to high production costs.

### SUMMARY

Exemplary embodiments provide a method of treating lignocellulose-based biomass to separate a high-purity cellulose, which is suitable for saccharification. The method provides high efficiency when utilizing a recycled solvent.

In one aspect, there is provided a method of pretreating lignocellulose-based biomass including: extracting lignin from biomass by adding a solvent for dissolving lignin to the lignocellulose-based biomass including lignin, hemicellulose and cellulose; and extracting the cellulose and/or hemicellulose by adding an ionic liquid to the remaining biomass after extracting the lignin.

In another aspect, a method of producing biofuel is provided. The method includes saccharifying hemicellulose and/or cellulose extracted from the pretreated lignocellulosic biomass to yield a monosaccharide by utilizing a hydrolase or a hydrolysis catalyst thereto.

### BRIEF DESCRIPTION OF THE DRAWINGS

Example embodiments are described in further detail below with reference to the accompanying drawings. It should be understood that various aspects of the drawings may have been exaggerated for clarity:

FIG. 1 is a schematic diagram of a structure of lignocellulose;

FIG. 2 is a schematic diagram showing a structural change in lignocellulose according to pretreatment (fractionation);

FIG. 3 is a flowchart showing an exemplary embodiment of a pretreatment process according to the disclosure;

FIG. 4 is a flowchart showing an exemplary embodiment of a process of producing biofuel according to the disclosure;

FIG. 5 is a photograph of biomass taken after the pretreatment process according to Experimental Example 1;

FIG. 6 shows glucose concentration measured after a 72-hour saccharification process at 50° C. according to Experimental Example 2;

FIG. 7 shows volumetric productivity of ethanol according to Experimental Example 4;

FIG. 8 shows glucose concentration versus the number of times that an ionic liquid is recycled according to Comparative Example 1 in Experimental Example 5;

FIG. 9 shows glucose concentration versus the number of times that an ionic liquid is recycled according to Example 1 in Experimental Example 5;

FIG. 10 is a graph showing relative results according to FIGS. 8 and 9; and

FIG. 11 shows a XRD pattern for various ionic liquids according to Experimental Example 6.

### DETAILED DESCRIPTION

Hereinafter, advantages, features and methods for embodying the disclosed concept will be described more fully with reference to the detailed descriptions of the following example embodiments and the accompanying drawings. However, it should be understood that the disclosed concept is not limited to the described example embodiments, and thus may be embodied in various forms.

The exemplary embodiments of the disclosure may, however, may be embodied in many different forms, and should not be construed as being limited to the embodiments set forth herein. Rather, these embodiments are provided so that this disclosure will be thorough and complete and will fully convey the concept of the disclosure to those skilled in the art, and the exemplary embodiments of the disclosure do not limit the scope of the claims. Like reference numerals refer to like elements throughout the specification.

It will be understood that when an element or layer is referred to as being “on” or “connected to” another element or layer, the element or layer can be directly on or connected to another element or layer or intervening elements or layers. In contrast, when an element is referred to as being “directly on” or “directly connected to” another element or layer, there are no intervening elements or layers present. As used herein, the term “and/or” includes any and all combinations of one or more of the associated listed items.

As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the disclosure are described herein with reference to cross-section illustrations that are schematic illustrations of idealized embodiments (and intermediate structures) of the disclosure. As such, variations from the shapes of the illustrations as a result, for example, of manufacturing techniques and/or tolerances, are to be expected. Thus, embodiments of the disclosure should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the claims.

1. Method of Pretreating Lignocellulose-Based Biomass

Generally, in lignocellulose, lignin and hemicellulose are linked to each other by covalent bonds, and the hemicellulose is linked to cellulose by hydrogen bonds, as shown in FIG. 1. Overall, the lignocellulose has a linear shape of cellulose microfibril, which is surrounded by hemicellulose via hydrogen bonds. Here, the hemicellulose is also surrounded by lignin via covalent bonds.

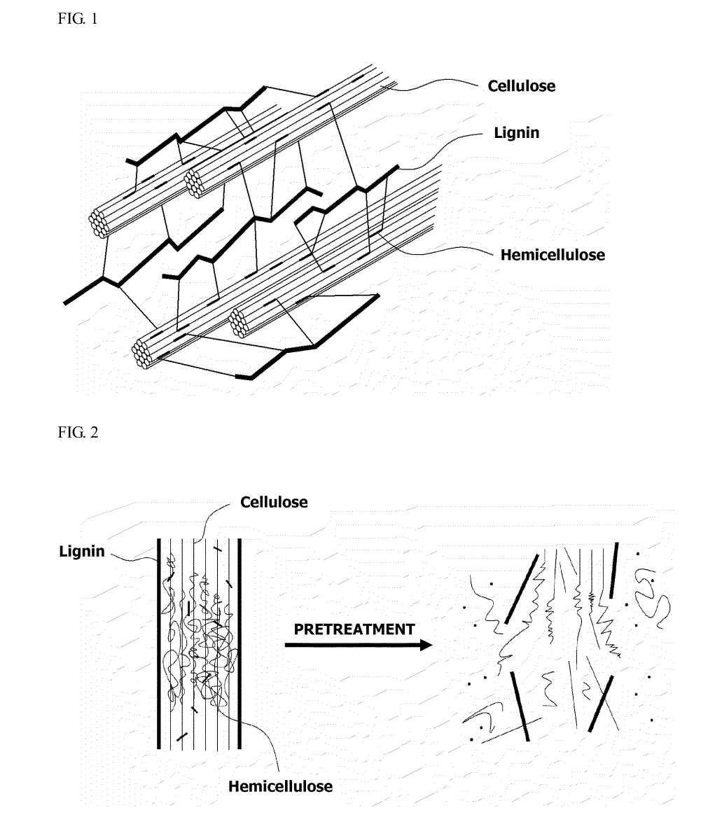

As shown in FIG. 2, the bonds between lignin, cellulose and hemicellulose may become weakened by pretreatment of the lignocellulose.

Conventionally, pretreated biomass still includes lignin and hemicellulose as well as cellulose, so that there are many impurities after saccharification and fermentation. Particularly, when the pretreated biomass includes lignin, a degradation product of lignin, usually a phenolic compound, acts as an inhibitive material in the saccharifying and fermenting process. Accordingly, there is a need for an additional process to separate or fractionate a specific component.

According to an exemplary embodiment, a method of pretreating lignocellulose-based biomass includes extracting lignin, and extracting cellulose and/or hemicellulose.

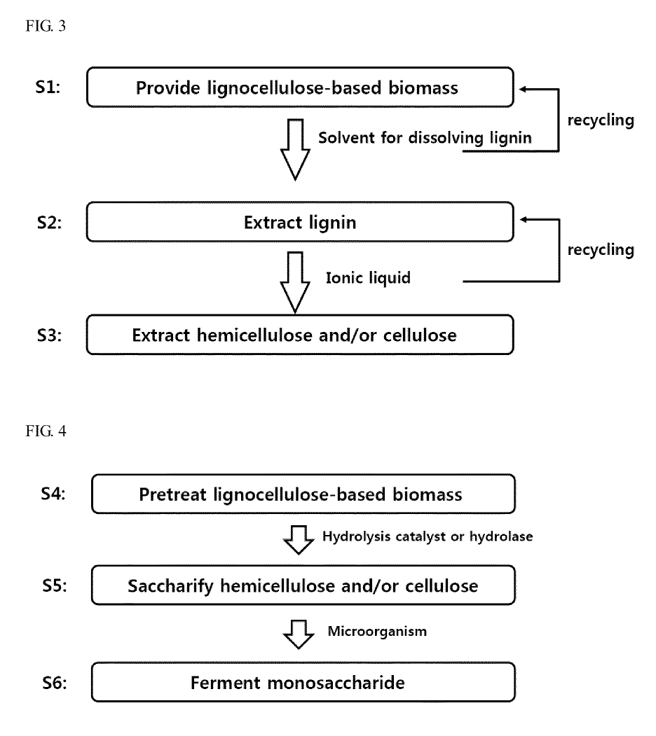

FIG. 3 is a flowchart showing a method of fractionating lignocellulose-based biomass according to an exemplary embodiment. Referring to FIG. 3, the method includes: providing lignocellulose-based biomass (S1); extracting lignin from the biomass by adding a solvent for dissolving lignin to the lignocellulose-based biomass (S2); and extracting cellulose and/or hemicellulose by adding an ionic liquid to the remaining biomass after extracting the lignin (S3).

According to the method described above, because the hemicellulose or cellulose is extracted after extracting the lignin from the lignocellulose-based biomass, production of any material that may inhibit the saccharification and fermentation process may be minimized. This method provides for a high-purity product that can be obtained in high yield. In addition, the disclosed method may be performed under milder conditions than conventional methods.

Therefore, when saccharification is performed by the method described above, the required amount of hydrolase or a hydrolysis catalyst, which takes a large portion of production costs, may be reduced, and the reaction rate may be increased, thereby enhancing saccharification efficiency. Further, the disclosed method is more economical since the needed amount of fermentation yeast may be reduced during fermentation.

Furthermore, the lignin concentration is low in the ionic liquid which is collected after the pretreatment, so that the purity of the ionic liquid is very high, and thus efficiency is high even when the ionic liquid is recycled. Accordingly, the utilization of relatively high-cost ionic liquid may be maximized, which is useful in commercial practice.

The lignocellulose-based biomass may be provided as a pellet or chip. A source of the lignocellulose-based biomass may be, but is not limited to, rice straw, hard wood, soft wood, herbs, recycled paper, waste paper, wood chips, pulp and paper wastes, waste wood, thinned wood, cornstalk, chaff, wheat straw, sugar cane stalk, bagasse, agricultural residual products, agricultural wastes, excretions of livestock, or mixtures thereof.

The method of providing biomass is not particularly limited, and thus the biomass may be continuously or discontinuously provided. When the biomass is continuously provided, a provider, a reactor, and a separator may be installed in one apparatus, and thus after extraction of each component, the solid component remaining in the reactor may be transferred to the separator and biomass may be provided from the provider at the same time. A continuous provider may be a percolation apparatus or an extruder, but the disclosed concept is not limited thereto. When biomass is discontinuously provided, once a reactor is filled with biomass, each component is extracted according to the above-described fractionating method, and a solid biomass component in the reactor is removed. Then, for subsequent processes, the reactor may be filled again with biomass.

The solvent for dissolving lignin may be a solvent capable of dissolving at least about 50 wt % of lignin, or a solvent capable of removing at least about 65 wt % of lignin. The solvent should not over-degrade the cellulose and hemicellulose under given conditions.

In one example, the solvent for dissolving lignin may be a basic solvent having pH of at least 10, or in the range from about pH 10 to about 13. Examples of the basic solvents may be, but are not limited to, aqueous ammonia, sodium hydroxide (NaOH), calcium hydroxide (Ca(OH)₂), sodium sulfate (Na₂SO₄) and mixtures thereof. In another example, the solvent for dissolving lignin may be an organic solvent such as ammonia, ethanol, butanol, methanol, acetone, ethylacetate or methylacetate, which are liquids easily fractionated by distillation due to their low boiling point. In addition, an oxygen donor such as H₂O₂ may be added to increase the effect of delignification.

The concentration of the basic solvent is not particularly limited, but a high concentration of the basic solvent may result in an increase in production costs and a decrease in stability due to increased vapor pressure, corrosion of the apparatus, and environmental contamination. In consideration of these problems, the concentration of the basic solvent may be about 5 to about 30% by weight of the basic solvent or about 10 to about 15% by weight of the solvent.

The residence time of the solvent for dissolving lignin in a reactor may be about 1 minute to about 1 hour, or about 5 to about 40 minutes. In some cases, the solvent for dissolving lignin may be recycled in a subsequent reaction after extracting lignin therefrom, and evaporating the extracted lignin through recirculation.

Conventional pretreatment of biomass may be performed at a high temperature to separate the non-degradable lignin component. For example, conventional fractionation, usually steam explosion, is performed at a high pressure and a high temperature of about 180 to about 250° C. However, when the pretreatment is performed at a high temperature, over-degradation of the hemicellulose into furfural or degradation of the cellulose into hydroxyl furfural occurs, resulting in a decrease in yield. Moreover, conventional fractionation is not economical because of high energy consumption.

Alternatively, in the disclosed embodiment, lignin is extracted first, so that the method may be performed under relatively mild conditions. For example, the process of extracting lignin may be performed at about 90 to about 110° C. for about 0.1 to about 10 hours, and the process of extracting cellulose may be performed at about 80 to about 150° C. for about 0.1 to about 20 hours. To maintain a solid-liquid reaction, a reaction pressure may be adjusted to about 150 to about 280 psig, or about 170 to about 230 psig.

The extracted lignin may be subjected to cooling or thermal exchange to increase its yield rate. A yield rate of the extracted lignin may be at least 50 wt %, or at least 65 wt % based on the total weight of lignin originally present in the biomass. This level of extraction will minimize any inhibition effect on the enzymatic saccharification process. The lignin is a hydrophobic complex polymer with a high molecular weight and includes a large quantity of aromatic compounds due to polymerized methoxylated coumaryl alcohol, coniferyl alcohol or sinaphyl alcohol. Thus, the extracted lignin may be used as fuel for a steam or electricity-generating boiler without further treatment, or may be utilized for its phenolic content by degradation of the lignin.

After the extraction of lignin, hemicellulose and/or cellulose may be extracted.

In exemplary example, the extraction of hemicellulose and/or cellulose may be performed by adding an ionic liquid to the remaining biomass after extracting the lignin.

The ionic liquids refer to liquids consisting of ions only, and among them, ionic liquids existing in a liquid phase at room temperature are called room temperature ionic liquids. Generally, the ionic liquid consists of a large-sized cation usually having nitrogen, and a smaller-sized anion. Because of the disparity in size between the cation and anion, the lattice energy of the compound is decrease resulting in a less crystalline structure with a low melting point.

Exemplary examples of the ionic liquid may include at least one of the compounds expressed by the following Formula (1):

[A]⁺[B]⁻ (1)

In Formula (1), [A]⁻ is selected from the group consisting of

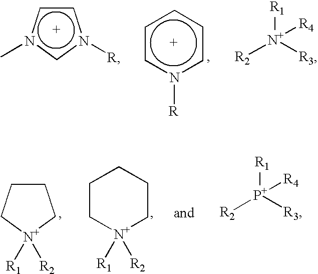

wherein R, R₁, R₂, R₃ and R₄ are each independently selected from the group consisting of hydrogen, C₁-C₁₅ alkyls, and C₂-C₂₀ alkenes, and the alkyl or alkene may be substituted by a substituent selected from the group consisting of sulfone, sulfoxide, thioester, ether, amide, hydroxyl and amine.

[B]⁻ is selected from the group consisting of Cl⁻, Br⁻, I⁻, OH⁻, NO₃⁻.SO₄²⁻, CF₃CO₂⁻, CF₃SO₃⁻, BF₄⁻, PF₆⁻, (CF₄SO₂)₂N⁻, AlCl₄⁻ and Cl⁻/AlCl₃ (How does this differ from AlCl₄⁻).

Examples of the compounds may include 1-butyl-3-methyl imidazolium tetrachloroaluminate, 1-ethyl-3-methyl imidazolium tetrachloroaluminate, 1-ethyl-3-methyl imidalzolium hydrogensulfate, 1-butyl-3-methyl imidazolium hydrogensulfate, methylimidazolium chloride, 1-ethyl-3-methyl imidazolium acetate, 1-butyl-3-methyl imidazolium acetate, tris-2(hydroxyl ethyl)methylammonium methylsulfate, 1-ethyl-3-methyl imidazolium ethylsulfate, 1-ethyl-3-methyl imidazolium methanesulfonate, methyl-tri-n-butylammonium methylsulfate, 1-butyl-3-methyl imidazolium chloride, 1-ethyl-3-methyl imidasolium chloride, 1-ethyl-3-methyl imidazolium thiocyanate, 1-butyl-3-methyl imidazolium thiocyanate, 1-aryl-3-methyl imidazolium chloride, and mixtures or complexes thereof, but the disclosed concept of utilizing ionic liquids is not limited to the disclosed species.

The ionic liquid may be commercially available, and may include Basionic™ AC 01, Basionic™ AC 09, Basionic™ AC 25, Basionic™ AC 28, Basionic™ AC 75, Basionic™ BC 01, Basionic™ BC 02, Basionic™ FS 01, Basionic™ LQ 01, Basionic™ ST 35, Basionic™ ST 62, Basionic™ ST 70, Basionic™ ST 80, Basionic™ VS 01, and Basionic™ VS 02, but the disclosed species is not limited thereto.

Alternatively, the compound may be 1-ethyl-3-methyl imidazolium hydrogensulfate of the following structural formula(2), 1-ethyl-3-methyl imidazolium acetate of the following structural formula(3), 1-ethyl-3-methyl imidazolium chloride of the following structural formula(4), or 1-n-butyl-3-methyl imidazolium chloride of the following structural formula(5):

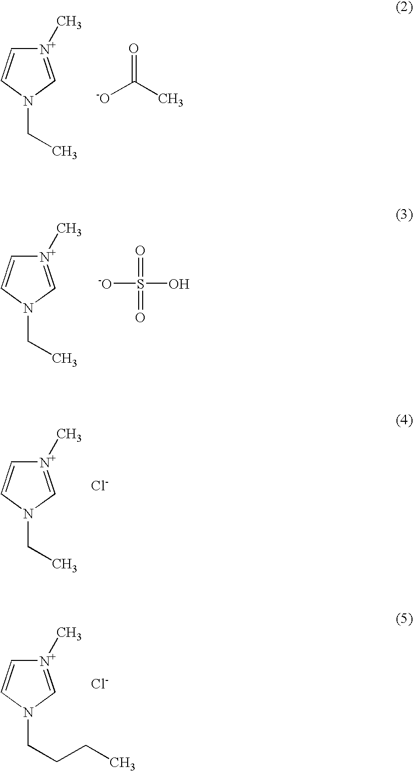

Some researchers hypothesize that the ionic liquid hinders the formation of hydrogen bonds between the hydroxyl groups of the cellulose. In this process, the anion binds to the hydrogen in the hydroxyl group of the cellulose, and the cation binds to oxygen in the hydroxyl group of cellulose, thereby dissolving the cellulose (R. C. Remsing, R. P. Swatloski, R. D. Rogers, and G Moyna, Chem. Commun., 1271, 2006).

Other researchers found that dissolution is completed in a molar ratio of glucose to an ionic liquid of 1:4, that is, a molar ratio of OH groups of glucose to an anion of an ionic liquid of 5:4, and two OH groups bind to one Cl anion when [B] is Cl. (T. G. A. Youngs, C. Hardacre, and J. D. Holbrey, J. Phys. Chem. B, 111, 13765, 2007).

In addition, other researchers found that an ionic liquid is effective in dissolving lignin as well as cellulose (D. A. Fort, R. C. Remsing, R. P. Swatloski, P. Moyna, G. Moyna, and R. D. Rogers, Green Chem., 9, 63, 2007).

However, ionic liquids cost about $450 per kg, which means it is a solvent that is about 2000 times as expensive as an aqueous ammonia solution, which costs about $0.2 per kg. Meanwhile, because ionic liquids are very stable and have a high boiling point, they are easily recovered for recycling. Thus, methods of recovering and recycling the ionic liquid can potentially overcome their economic disadvantage.

However, when the pretreatment process utilizes only an ionic liquid, lignin is contained in the pretreated biomass, and the ionic liquid collected after the pretreatment also contains the lignin. When the ionic liquid containing lignin is recycled, the dissolution efficiency of the cellulose is significantly decreased.

In contrast, as disclosed herein, the lignin is first extracted from lignocellulose-based biomass, followed by extraction of the cellulose and hemicellulose using an ionic liquid. Thus, the lignin is not substantially contained in the ionic liquid collected after extraction, so that the decrease in dissolution efficiency is minimized.

Accordingly, the ionic liquid may be effectively recycled after the extraction of cellulose.

An amount of the ionic liquid added herein is not particularly limited, but may be about 5 to 20 times higher than the content of the solid component remaining after lignin extraction.

In the biomass pretreated according to various example embodiments, a main component is cellulose pretreated to facilitate its reactivity with enzymes and to minimize the content of the lignin component. Thus, there may be almost no inhibition of the saccharification process, the amount of enzyme used may be remarkably decreased, and the monosaccharide yield may be ultimately increased by increasing the reaction rate.

2. Method of Producing Biofuel

In another embodiment, a method of producing biofuel is disclosed and includes saccharifying the lignocellulose-based biomass pretreated according to the above-described embodiments.

As described above, for pretreatment of the biomass, a solvent for dissolving lignin and an ionic liquid are sequentially used, and the ionic liquid transforms the cellulose from a crystalline phase into an amorphous phase. Thus, a hydrolysis catalyst or a hydrolase may easily react with the cellulose substrate in the saccharification process, resulting in an increase in saccharification efficiency.

The saccharification may include enzymatic saccharification with a hydrolase, treating with weak sulfuric acid, and treating with microorganisms capable of producing the hydrolase.

In exemplary example, the saccharification may be performed with a hydrolase or a hydrolysis catalyst.

The hydrolase may include cellulase, α-amylase, glucoamylase, endoglucanase, exoglucanase, xylanase, β-glucosidase, α-agarase, β-agarase I, β-agarase II, β-galactosidase, neoagarobiose, neoagarotetraose, neoagarohexaose, α-neoagrobiose hydrolase, or a mixture or complex thereof, but the disclosed concept is not limited to these hydrolases.

The hydrolysis catalyst may include H₂SO₄, HCI, HBr, HNO₃, CH₃COOH, HCOOH, HClO₄, H₃PO₄, Para-toluene sulfonic acid (PTSA), or a mixture or complex thereof, but the disclosure is not limited to these catalysts.

As described above, when the saccharification is performed through a predetermined pretreatment to facilitate reactivity of the hydrolysis catalyst or the hydrolase toward the biomass, the use of the hydrolysis catalyst or hydrolase may be decreased by at least 50%. For example, when the biomass is pretreated using sulfuric acid according to conventional art, about 10 to 16 filter paper unit (FPU)/g of enzyme may be used, but according to an example embodiment, the same or a higher saccharification rate may be achieved using only about 5 to 8 FPU/g of enzyme.

Saccharification time may also be decreased by at least 70%. For example, when the biomass is pretreated with sulfuric acid according to conventional art, the saccharification time is about 72 hours, but according to an example embodiment, at least 90% saccharification may be achieved in a 24 to 65-hour saccharification time, and preferably, but not necessarily, in a 24 to 48-hour saccharification time. Thus, the saccharification efficiency may be significantly increased.

Meanwhile, according to recent research, when an ionic liquid is used as a reaction medium for saccharification of cellulose using sulfuric acid as a catalyst, a yield of glucose may be increased (C. Li and Z. K. Zhao, Adv. Synth. Catal., 349, 1847 2007).

Another researcher has reported that when cellulose is treated with an ionic liquid, and hydrolyzed with cellulose (from T. reesei), a yield of glucose may be increased by about 2 times (A. P. Dadi, S. Varanasi, and C. A. Schall, Biotech. Bioeng., 95, 904, 2006).

In exemplary embodiment, an ionic liquid may be added to the saccharification process. Examples of the ionic liquid may include those described above.

The monosaccharide may be a hydrolyte of lignocellulose-based biomass. For example, the hydrolyte may include at least one selected from the group consisting of glucose, galactose, a galactose derivative, 3,6-anhydrogalactose, fucose, rhamnose, xylose, arabinose and mannose, but the disclosure is not limited to these hydrolytes. Alternatively, the hydrolyte may include glucose, or a mixture of glucose and galactose.

The biofuel may be an alcohol such as ethanol or butanol, an alkane-based compound, a C₃ to C₆-based chemical source or an organic acid, but the disclosure is not limited to these biofuels.

In exemplary embodiment, the method of producing biofuel may further include fermentation for producing alcohol by fermenting the monosaccharide obtained in the saccharification process.

FIG. 4 is a flowchart showing a method of producing biofuel according to an example embodiment. According to FIG. 4, the biofuel may be produced through pretreatment (S4), saccharification (S5) and fermentation (S6).

For example, the saccharification (S5) may be performed by filling cellulose and a saccharification enzyme in a saccharification reaction vessel and saccharifying the cellulose at an optimum temperature of the saccharification enzyme to produce a saccharification liquid, and filling microorganisms in a fermentor and providing the saccharification liquid to perform fermentation at an optimum temperature.

The fermentation (S6) is performed by fermenting a monosaccharide such as a 5- or 6-carbon saccharide by a microorganism to convert the monosaccharide into ethanol, as shown in the following formulae:

C₆H₁₂O₆→2C₂H₅OH+2CO₂

3C₅H₁₀O₅→5C₂H₅OH+5CO₂

When the biomass is treated as described above, volumetric productivity of ethanol (g/L/h) may be at least 95%. Here, the volumetric productivity of ethanol refers to time to produce ethanol in maximum concentration by consuming a given substrate.

The microorganism for fermentation may differ from the kind of the monosaccharide and may include various microorganisms well known in the art.

Examples of the microorganism may include, but are not limited to, Saccharomyces cerevisiae, Klebsiella oxytoca P2, Brettanomyces curstersii, Saccharomyces uvzrun, Candida brassicae, Sarcina ventriculi, Zymomonas mobilis, Kluyveromyces marxianus IMB3, Clostridium acetobutylicum, Clostridium beijerinckii, Kluyveromyces fragilis, Brettanomyces custersii, Clostriduim aurantibutylicum and Clostridium tetanomorphum.

Conditions for the fermentation are not particularly limited, and the fermentation may be performed by stirring a culture under the following conditions: an initial glucose concentration of about 2 to about 30% (w/v), a temperature of about 25 to about 37° C., a pH of about 5.0 to about 8.0, and a stirring rate of about 100 to about 250 rpm.

In addition, the saccharification and fermentation may be performed in separate reaction vessels through a separate hydrolysis and fermentation (SHF) process, or in one reaction vessel through a simultaneous saccharification and fermentation (SSF) process.

The SHF process may be performed under optimized conditions for respective saccharification and fermentation, but may create inhibition of enzymatic hydrolysis between an intermediate product and a final product. Thus, more enzymes are needed to overcome this problem, which is uneconomical. For example, when an intermediate product, cellobiose, is converted into a final product, glucose, in the saccharification of cellulose, glucoses are accumulated, thereby inducing inhibition of the hydrolysis between the intermediate product and the final product, resulting in termination of the reaction.

In comparison, in the SSF process, as soon as glucose is produced in the saccharification process, yeast consumes the glucose through fermentation and thus glucose accumulation in a reaction vessel can be minimized. As a result, inhibition driven by a final product, which can occur in the SHF process, can be prevented, and hydrolysis mediated by a hydrolase can be enhanced. Further, the SSF process can reduce production costs due to low equipment costs and low input of enzyme, and also lessen a risk of contamination due to ethanol present in the reaction vessel.

Additional operations and/or other processes may be selected by those skilled in the art as occasion demands. For example, purification of a fermented liquid yielded by the fermentation according to the known method in the art may be added.

Hereinafter, the disclosed concept will be described with reference to examples of the disclosed concept.

### EXAMPLE 1

1-1. Pretreatment of Lignocellulose

Pretreatment is performed according to standard methods of National Renewable Energy Laboratory (NREL), USA. Domestic rice straw (RS), produced in 2007, which contains 35 to 40 wt % cellulose of a dry cell weight, is crushed into 2- to 5-mm particles using a crusher.

10% aqueous ammonia is added at a volume of about 10 times per 1 g of the crushed rice straw for a 6-hour reaction at 100° C., and then cooled. The extracted lignin is then separated from the remaining solid component to form a first solid component.

1-n-butyl-3-methylimidazolium chloride (BmimCl) is added at a volume of about 20 times with respect to the first solid component (Biomass: IL=1:20) for an 18-hour reaction at 130° C.

An antisolvent such as ethanol is added to the above-treated solution to induce precipitation and then filtered to yield second solid component, followed by drying the second solid component for saccharification.

1-2. Enzymatic Saccharification and Fermentation of Pretreated Biomass

1.5 L of celluclast (Novozyme), Novozyme 188 (Novozyme) and 28.5 ml of distilled water are added per 1 g of the second solid component (cellulose) for a 72-hour reaction using an enzyme at pH 4.8 and at 50° C., resulting in producing glucose and xylose. The content of the enzyme used herein is in the ratio of 12:1.2 (FPU: CPU)

The yielded enzyme reaction mixture is centrifuged at room temperature at 4000 rpm for 10 minutes to harvest a supernatant in a triangle flask, sterilized at 121° C. for 15 minutes and then cooled. Then, the cooled supernatant is inoculated with a culture of S. cerevisiae having an optical density (600 nm) of about 5 in an inoculum concentration of 10 to 20% for 24-hour incubation at 30° C. at 150 rpm. After about 5 hours of inoculation, an opening of the flask is sealed to give an anaerobic condition.

### COMPARATIVE EXAMPLE 1

Ionic liquid (Please list liquid) is added at a volume of 20 times per 1 g of crushed rice straw, and pretreated at 130° C. for 48 hours. Subsequently, an antisolvent such as ethanol is added to the pretreated solution to induce precipitation and filtered, yielding a first solid component, which is then dried for saccharification and fermentation through the method according to Example 1-2.

Measurement of Concentrations of Glucose and Ethanol

Concentrations of glucose produced by an enzyme reaction and ethanol produced by fermentation are measured using HPLC. First, diluted samples are filtered using a 0.2 μm filter, and a content of glucose or ethanol is analyzed using HPLC (Shimadzu, Japan). A 20 μl of sample is injected into an HPLC having a 4.6×10 mm guard column (Bio-Rad, USA) and a 4.6×150 mm column (Aminex 87HP, Bio-Rad, USA), and distilled water is used as a transfer phase. The concentration of glucose or ethanol is measured using an RI detector at a flow rate of 0.6 ml/min and at 60° C.

### EXPERIMENTAL EXAMPLE 1

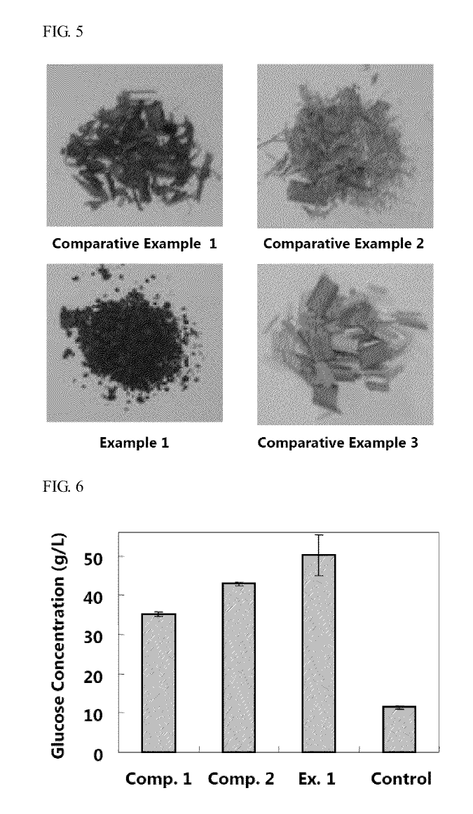

After pretreatment is completed according to each of Example 1 and Comparative Examples 1, each pretreated biomass is photographed, which is shown in FIG. 5. Referring to FIG. 5, it can be seen that the biomass sequentially treated with aqueous ammonia and an ionic liquid according to Example 1 exhibits a remarkable morphological change.

### EXPERIMENTAL EXAMPLE 2

### Measurement of Concentration of Glucose

FIG. 6 shows the concentration of glucose after 72-hour saccharification at 50° C. Referring to FIG. 6, it can be seen that Example 1 exhibits at least 40 wt % increase in yield in enzyme reaction, compared to Comparative Example 1.

### EXAMPLE 2

After pretreatment is completed according to Example 1-1, biomass is saccharified and fermented through the same method described in Example 1-2, except that the content of enzyme is set to 9:0.9 (FPU: CPU).

### EXAMPLE 3

After pretreatment is completed according to Example 1-1, biomass is saccharified and fermented through the same method described in Example 1-2, except that the content of enzyme is set to 6:0.6 (FPU: CPU).

### EXPERIMENTAL EXAMPLE 3

### Comparison of Saccharification Rates According to Amount of Enzyme Used

To compare saccharification rates according to the amount of enzyme used in Comparative Example 1, control group and Examples 1 to 3, a saccharification rate is measured in each sample after 14, 24 and 48 hours of reaction, and the results are shown in Table 1.

TABLE 1

| | | Enzyme | | | |
|---|---|---|---|---|---|
| | Treatment | Content | Saccharification | Saccharification | Saccharification |
| | Condition | (FPU:CPU) | Rate (12 h, %) | Rate (24 h, %) | Rate (48 h, %) |
| C. Example 1 | Only IL | 12:1.2 | 22 | 74 | 78 |
| Control group | none | 12:1.2 | 6 | 20 | 21 |
| Example 1 | NH₄OH + IL | 12:1.2 | 32 | 90 | 95 |
| Example 2 | | 9:0.9 | — | 88 | 99 |
| Example 3 | | 6:0.6 | — | 86 | 95 |

(*IL refers to ionic liquid above)

Referring to Table 1, volumetric productivities of glucose (g/l/h) after initial 12 hours of reaction, are about 0.6 g/L/h (saccharification rate: 6%) for the untreated rice straw (control), about 2.2 g/L/h (saccharification rate: 22%) for Comparative Example 1, and about 3.2 g/L/h (saccharification rate: 32%) for Example 1. It can be seen that when the biomass is pretreated according to Example 1, volumetric productivity of ethanol is increased about 5 times greater than control group, and about 1.5 times greater than Comparative Example 1.

In addition, although the contents of enzyme used in Examples 2 and 3 are about 25 and 50% lower than that used in Comparative Example 1, both Examples 2 and 3 are at least 95% increased in saccharification rate within 48 hours.

### EXPERIMENTAL EXAMPLE 4

### Measurement of Volumetric Productivity of Ethanol

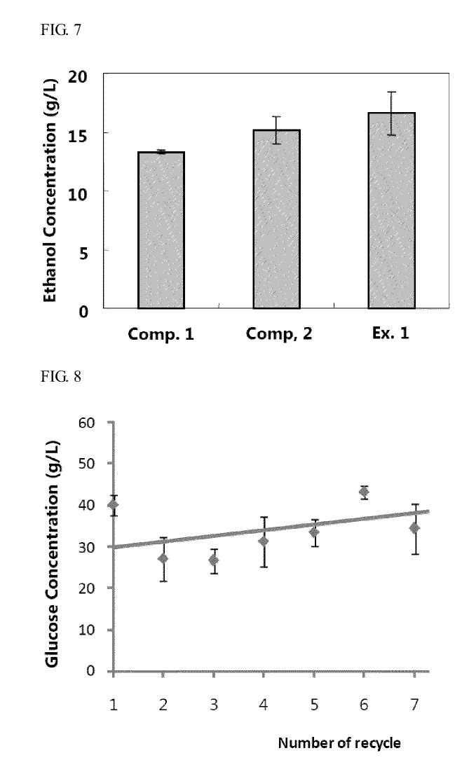

Volumetric productivities of ethanol are measured in Example 1 and Comparative Examples 1, and the result is shown in FIG. 7. It can be seen that the yield of ethanol, compared to glucose, is maintained in the range from about 40 to 45%, and the concentration of ethanol produced in Example 1 is about 25% higher than Comparative Example 1. As a result, it can be concluded that when biomass is sequentially pretreated with aqueous ammonia and an ionic liquid, the yield of bioethanol may be ultimately increased.

### EXPERIMENTAL EXAMPLE 5

### Yield according to Recycle of Ionic Liquid

After pretreating biomass with an ionic liquid in Comparative Example 1 and Example 1, respectively, the pretreated biomass is recovered with an antisolvent in a liquid phase, and the ionic liquid is separated from the antisolvent through vacuum distillation for recycling in a reaction vessel containing the biomass. Concentrations of glucose are measured according to the number of times that the ionic liquid is recycled, which are shown in FIGS. 8 and 9.

Referring to FIGS. 8 and 9, at the 7th recycle, when the ionic liquid is recovered from the biomass treated according to Example 1, the glucose concentration is 54 g/L, whereas when the only ionic liquid is recovered as described in Comparative Example 1, the glucose concentration is about 34 g/L. As a result, it can be seen that volumetric productivity of glucose in Example 1 is increased 63%, compared to Comparative Example 1.

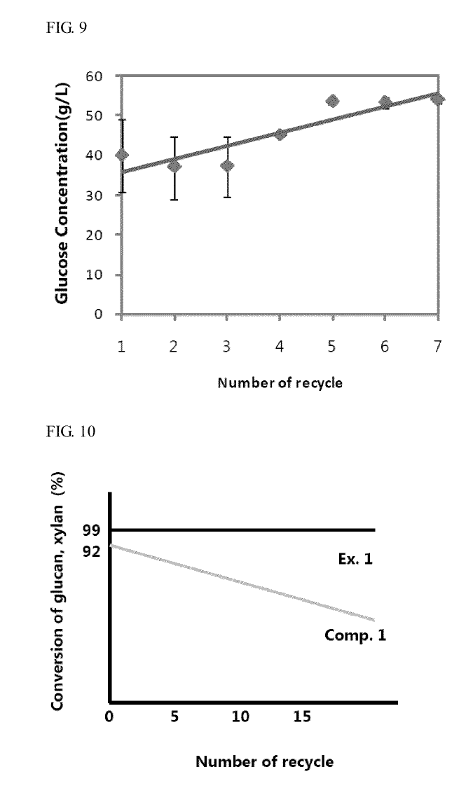

We believe that the reason that the glucose concentration is increased when the ionic liquid is recycled in FIGS. 8 and 9 is that a saccharide component such as cellulose remains in the recycled ionic solvent. FIG. 10 shows comparison of saccharification rates according to the number of times that the ionic liquid is recycled in Example 1 and Comparative Example 1.

Referring to FIG. 10, it can be clearly seen that Comparative Example 1 shows a decrease in saccharification rate as the number of times that the ionic liquid is recycled is increased, whereas Example 1 shows almost uniform saccharification rates even when the number of times that the ionic liquid is recycled is increased.

### EXAMPLE 4

Pretreatment is performed by the same method as Example 1-1, except that 1-ethyl-3-methylimidazolium chloride is used instead of 1-n-butyl-3-methylimidazolium chloride.

### EXAMPLE 5

Pretreatment is performed by the same method as Example 1-1, except that 1-ethyl-3-methylimidazolium sulfate is used instead of 1-n-butyl-3-methylimidazolium chloride.

### EXAMPLE 6

Pretreatment is performed by the same method as Example 1-1, except that 1-ethyl-3-methylimidazolium acetate is used instead of 1-n-butyl-3-methylimidazolium chloride.

### EXPERIMENTAL EXAMPLE 6

### XRD Pattern According to the Type of Ionic Liquid

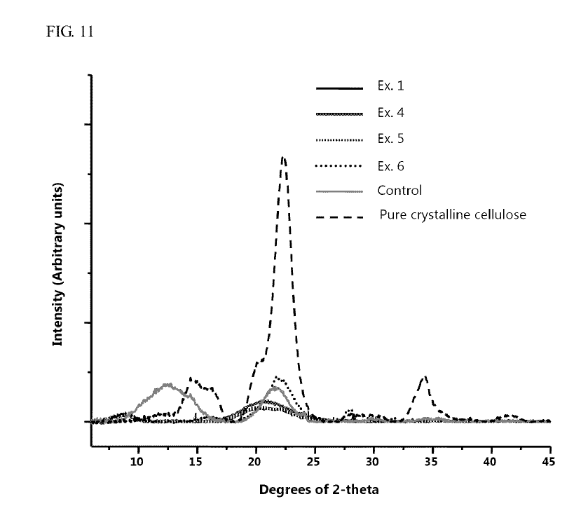

XRD patterns of biomasses pretreated according to Examples 1, and 4 to 6, control biomass and pure crystalline cellulose are determined, and the results are shown in FIG. 11.

Referring to FIG. 11, it can be seen that when pretreatments are performed according to Examples, crystallinity is lower than the control group and the pure crystalline cellulose. Particularly, Example 1 shows the lowest crystallinity, and therefore it can be seen that the conversion rate is increased when an enzyme is treated.

In a method of pretreating lignocellulose-based biomass according to example embodiments, lignin is first extracted, and hemicellulose and/or cellulose are extracted, obtaining a high-purity pretreatment product. When saccharification and fermentation are performed using this method, a high-efficiency and high-purity material can be obtained. In addition, almost no impurity is included in solvents used in pretreatment, so that high efficiency can be exhibited when the solvents are recycled, which is very effective in economical and industrial aspects.

While example embodiments have been disclosed herein, it should be understood that other variations may be possible. Such variations are not to be regarded as a departure from the spirit and scope of example embodiments of the present application, and all such modifications as would be obvious to one skilled in the art are intended to be included within the scope of the following claims.

What is claimed is:

1. A method of pretreating lignocellulose-based biomass, comprising:
extracting lignin from a lignocellulose-based biomass by adding a solvent for dissolving the lignin from the lignocellulose-based biomass which includes lignin, hemicellulose and cellulose; and
extracting the cellulose and/or hemicellulose by adding an ionic liquid to the remaining biomass after extracting the lignin.

2. The method according to claim 1, wherein the solvent for dissolving lignin is a basic solvent.

3. The method according to claim 2, wherein the basic solvent is at least one selected from the group consisting of aqueous ammonia, sodium hydroxide (NaOH), calcium hydroxide (Ca(OH)₂), sodium sulfate (Na₂SO₄) and combinations thereof

4. The method according to claim 3, wherein the basic solvent is aqueous ammonia.

5. The method according to claim 2, wherein the basic solvent has a concentration of about 5 to about 30 wt % based on the total weight of the solvent solution.

6. The method according to claim 1, wherein the lignin is extracted, and then the solvent is evaporated for recirculation.

7. The method according to claim 1, wherein the ionic liquid includes at least one compound expressed by Formula (1):
[A]⁻[B]⁻ (1)
wherein,
[A]⁻ is selected from the group consisting of 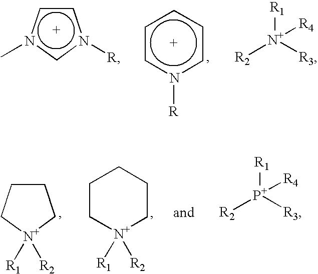
R, R₁, R₂, R₃ and R₄ are each independently selected from the group consisting of hydrogen, C₁-C₁₅ alkyls, and C₂-C₂₀ alkenes, the alkyl or alkene may be substituted by a substituent selected from the group consisting of sulfone, sulfoxide, thioester, ether, amide, hydroxyl and amine; and
[B]⁻ is selected from the group consisting of Cl⁻, Br⁻, I⁻, OH⁻, NO₃⁻.SO₄²⁻, CF₃CO₂⁻, CF₃SO₃⁻, BF₄⁻, PF₆⁻, (CF₄SO₂)₂N⁻, AlCl₄⁻ and Cl⁻/AlCl₃.

8. The method according to claim 7, wherein the compound of Formula (1) is selected from the group consisting of 1-butyl-3-methyl imidazolium tetrachloroaluminate, 1-ethyl-3-methyl imidazolium tetrachloroaluminate, 1-ethyl-3-methyl imidalzolium hydrogensulfate, 1-butyl-3-methyl imidazolium hydrogensulfate, methylimidazolium chloride, 1-ethyl-3-methyl imidazolium acetate, 1-butyl-3-methyl imidazolium acetate, tris-2(hydroxyl ethyl)methylammonium methylsulfate, 1-ethyl-3-methyl imidazolium ethylsulfate, 1-ethyl-3-methyl imidazolium methanesulfonate, methyl-tri-n-butylammonium methylsulfate, 1-butyl-3-methyl imidazolium chloride, 1-ethyl-3-methyl imidasolium chloride, 1-ethyl-3-methyl imidazolium thiocyanate, 1-butyl-3-methyl imidazolium thiocyanate, 1-aryl-3-methyl imidazolium chloride, and mixtures or complexes thereof.

9. The method according to claim 8, wherein the compound of Formula (1) is selected from the group consisting of 1-ethyl-3-methyl imidazolium hydrogensulfate, 1-ethyl-3-methyl imidazolium acetate, 1-ethyl-3-methyl imidazolium chloride, and 1-n-butyl-3-methyl imidazolium chloride.

10. The method according to claim 1, wherein the ionic liquid is recycled after extracting the cellulose and/or hemicellulose.

11. The method according to claim 1, wherein an amount of the added ionic liquid is about 5 to about 20 times greater than a solid component remaining after extracting the lignin.

12. The method according to claim 1, wherein the extraction of the lignin is performed at about 90 to about 110° C. for about 0.1 to about 10 hours.

13. The method according to claim 1, wherein the extraction of the cellulose is performed at about 80 to about 150° C. for about 0.1 to about 20 hours.

14. A method of producing a biofuel comprising saccharifying the cellulose and/or hemicellulose extracted from the lignocellulosic biomass pretreated by the method of claim 1 to yield a monosaccharide by adding a hydrolase or a hydrolysis catalyst for hydrolysis thereto.

15. The method according to claim 14, wherein the hydrolase is cellulase.

16. The method according to claim 14, wherein the hydrolase is used about 5 to about 8 FPU/g.

17. The method according to claim 14, wherein the saccharifying is performed for about 24 to about 65 hours.

18. The method according to claim 14, further comprising fermenting the monosaccharide yielded in the saccharifying to produce alcohol.

19. The method according to claim 18, wherein the fermenting is performed using Saccharomyces cerevisiae.

20. The method according to claim 14, wherein volumetric productivity of ethanol (g/L/h) is at least 95%.
